(19) United States
(12) Patent Application Publication
(10) Pub. No.: US 2009/0110661 A1
(43) Pub. Date: Apr. 30, 2009

(54) Novel Gene Therapy Approach For Treating The Metabolic Disorder Obesity

(75) Inventor: Sergey Musatov, New York, NY (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: NEUROLOGIX, INC., Fort Lee, NJ (US)

(21) Appl. No.: 12/261,451

(22) Filed: Oct. 30, 2008

Related U.S. Application Data
(60) Provisional application No. 61/001,011, filed on Oct. 30, 2007.
(60) Provisional application No. 60/984,984, filed on Nov. 2, 2007.

(51) Int. Cl. A61K 38/21; A61K 48/00; A61K 31/7052; A61P 3/04; A61K 38/28; A61K 38/16

(52) U.S. Cl. 424/85.6; 514/44; 514/12; 424/85.7; 514/3

(57) ABSTRACT

The present application relates to novel methods for treating obesity. Some aspects pertain to the use of gene therapy to treat diseases related to metabolic dysfunction, such as diabetes, obesity, high blood pressure, and atherogenic dyslipidemia. The present application also pertains to the use of vectors such as a recombinant adeno-associated virus (AAV) to deliver a gene that can increase or decrease expression of a therapeutic protein of interest, e.g., in cells in a specific region of the brain associated with metabolic dysfunction. The present application also discloses the use of vectors such as a recombinant adeno-associated virus for the delivery of small interference RNA's (siRNAs) capable of decreasing expression of a deleterious protein involved in the disorder. Other related aspects, including compositions related to such methods, are also disclosed.

### CROSS REFERENCE TO RELATED APPLICATIONS

The present application claims the benefit of U.S. Provisional Application No. 61/001,011 filed on Oct. 30, 2007, entitled “A Novel Gene Therapy Approach For Treating The Metabolic Disorder Obesity.” The entire contents of the provisional application are hereby incorporated herein by reference.

### FIELD OF INVENTION

This invention relates to novel methods for treating obesity. More specifically, the invention pertains to the use of gene therapy to treat diseases related to metabolic dysfunction, such as diabetes, obesity, high blood pressure, and atherogenic dyslipidemia. The invention also pertains to the use of vectors such as recombinant adeno-associated virus (AAV) to specifically deliver a gene capable of increasing or decreasing expression of a therapeutic protein of interest in cells in a specific region of the brain associated with metabolic dysfunction. The invention also pertains to the use of a vector for the delivery of small interference RNAs (siRNAs) capable of silencing expression of a deleterious protein involved in the disorder.

### BACKGROUND

Disorders of metabolic pathways play an important role in the progression of various disease processes. For example, diseases such as Type-II diabetes, hypertension, high cholesterol, atherogenic dyslipidemia have been identified as arising from disorders related to metabolism. The term “metabolic Syndrome” has been coined to refer to a cluster of conditions that occur together, and increase the risk for heart disease, stroke and diabetes. Having just one of these conditions such as increased blood pressure, elevated insulin levels, excess body fat around the waist or abnormal cholesterol levels increases the risk of the above mentioned diseases. In combination, the risk for coronary heart disease, stroke and diabetes is even greater.

Research into the complex underlying processes linking this group of conditions is ongoing. As the name suggests, metabolic syndrome is tied to the body's metabolism, and more likely to a condition called insulin resistance. Although, not all experts agree on the definition of metabolic syndrome or whether it even exists as a distinct medical condition, this collection of risk factors is becoming prevalent with an estimated 50 million Americans suffering from some form of metabolic disorder.

Obesity is a chronic disease manifested by an excess of fat mass in proportion to body size. Today, every third American is considered over-weight (Body Mass Index (BMI) >25 kg/m²), thus prompting the United States Centers for Disease Control and Prevention (CDC) to declare that obesity is reaching epidemic proportions (Cummings et al., Genetics and Pathophysiology of Human Obesity, Annu. Rev. Med., vol. 54, pg. 453, 2003). The importance of treating obesity is emphasized by the fact that this disease is either the underlying cause, or a risk factor, for developing diseases such as type 2 diabetes, congestive heart failure, osteoarthritis and sleep apnea among others.

Obesity is also linked to “metabolic Syndrome” which is a medical condition characterized by excess body fat, atherogenic dyslipidemia, elevated blood pressure and insulin resistance. Importantly, it has been shown that even a modest decrease in body weight (5-10% of initial body weight) may significantly improve conditions associated with the metabolic syndrome and decrease the risk factors for developing obesity-associated disease (Tuomilehto et al., Prevention of type 2 diabetes mellitus by changes in lifestyle among subjects with impaired glucose tolerance, New Engl. J. Med., vol. 344, pg. 1343, 2001; Knowler et al., Reduction in the incidence of type 2 diabetes with lifestyle intervention or metformin, New Engl. J Med., vol. 346, pg. 393, 2002; Franz et al., Evidence-based nutrition principles and recommendations for the treatment and prevention of diabetes and related complications, Diabetes Care, vol. 25, pg. 148, 2002). Additionally, treatment of obesity may be important from a mental health perspective due to the social stigma often attached to obese individuals in some cultures.

Obesity is caused by both genetic and environmental factors. Genetic causes of this abnormality can result from a single gene mutation in animals, but humans rarely develop obesity from a single gene mutation (Chaganon, et al., The Human Obesity Gene Map: The 1997 Update, Obes. Res. vol. 6, pg. 76, 1998). To develop treatments for obesity, studies delineating the pathophysiology of body weight regulation are vital. It has been found that fat serves not only as a reservoir for energy but also causes the secretion of substances involved in energy homeostasis. One such substance is “leptin” a hormone whose concentration in blood serum is related to the proportion of body fat. Leptin regulates body fat content and energy expenditure by influencing the brain. Mutations in the gene for leptin or its receptors have been identified as one possible cause for obesity. Leptin has also been implicated to be associated with hyperphagia, hyperinsulinemia, and insulin resistance (Prasad, et al., A Paradoxical Elevation of Brain Cyclo (his-pro) Levels in Hyperphagic Obese Zucker Rats, Brain Res. vol. 699, pg. 149, 1995). Initial studies focused on the use of leptin for treating obesity showed that common diet-induced obese mice were relatively insensitive to increased concentrations of systemic leptin. These studies also showed that the reduced sensitivity to systemically administered leptin arose either from a reduced transport of leptin to the brain, or due to inhibition of leptin-induced signal transduction to the hypothalamus. Thus, there is a need for therapy targeted towards the treatment of leptin-resistant obesity.

Another factor that has been demonstrated to play an important role in regulation of food intake and energy expenditure is the estrogen receptor α (ERα), and especially the estrogen receptors in the ventromedial nucleus (VMN) of the brain. Recent studies have shown the ERα modulates the glucose sensing function of the glucose responsive (GR) neurons, and that a disruption of ERα signaling can lead to an obese phenotype and the development of disorders related to the metabolic syndrome. Thus, there is a need for therapy targeted towards the treatment of metabolic disorders via restoration of ERα signaling to normal physiological levels.

Obesity is currently treated, with only limited success, by several different strategies. These strategies primarily involve “life-style” changes (e.g. diet and exercise), small molecule based pharmaceutical therapies or surgical removal of a portion of the stomach (gastric by-pass surgery). Additionally, weight loss stimulating melanocortin receptor binding peptides such as alpha-MSH are of limited use as pharmaceuticals due to the extremely short serum half-life of such peptides. In addition, drug treatment for obesity has been disappointing since almost all drug treatments for obesity were associated with undesirable side effects that contribute to the termination of their prolonged use as therapeutics. Available pharmacotherapies have included Sibutramine, Orlistat, fenfluramine and dexfenfluramine. Fenfluramine and dexfenfluramine were withdrawn from the market in 1997 because of associated cardiac valvulopathy (Connolly et al., Valvular Heart Disease Associated With Fenfluramine-Phentermine, New Engl. J. Med., vol. 337, pg. 581, 1997). Therefore, health care professionals continue to be reluctant to use pharmacotherapy in the management of obesity. Complimentary approaches to pharmacotherapy are therefore of great interest to the public. In this application, we propose a novel gene therapy approach for the treatment of obesity.

### SUMMARY OF THE INVENTION

The present invention is drawn to methods for treating a metabolic disorder. In some embodiments, at least a portion of a gene can be provided to increase or decrease expression of a therapeutic protein of interest to at least one cell. A vector can be used for delivering the gene. Expression of the therapeutic protein is increased or decreased in the transfected cells thereby treating the metabolic disorder.

Accordingly, one embodiment of the instant invention relates to treatments for metabolic disorders such as obesity, type-2 diabetes, hypertension and atherogenic dyslipidemia.

Another embodiment of the invention relates to providing a polynucleotide sequence that functions as at least one of a shRNA, a siRNA and a RNAi. Preferably, the polynucleotide comprises an estrogen receptor-alpha gene (ERα).

In one aspect, the therapeutic agent of the invention can be selected from the group consisting of Hip2, PGC1-alpha, and estrogen receptor-alpha (ERα). In some particular embodiments, the therapeutic protein is an estrogen receptor-alpha (ERα).

In other embodiments, the gene incorporated into the vector can comprise at least a portion of a gene from at least one of Hip2, PGC1-alpha, and ERα. In a particular embodiment, the incorporated gene is at least a portion of an ERα gene.

In some embodiments, the invention provides a method for using a vector (e.g., viral or non-viral) to deliver the gene encoding the desired protein into cells. A viral vector can be selected from a group consisting of adeno-associated viral vector, herpes simplex viral vector, parovirus vector and lentivirus vectors. In a preferred embodiment, the viral vector is an adeno-associated viral vector (AAV). In another embodiment, a non-viral vector can be a liposome-mediated delivery vector.

In yet another aspect, this invention discloses the use of recombinant adeno-associated virions having a cap-region from one type of AAV and a rep-region from a second type of AAV which is distinct from the first AAV. Such recombinant AAV's have the advantage of exhibiting modified tropism, (i.e., being highly selective with respect to the tissues it infects), as well as having a higher rate of transduction efficiency when compared to native AAV. A particularly favorable adeno-associated virion has a non-native capsid from AAV-1 and a rep-region from AAV-2.

In some embodiments, a vector is delivered to a desired region of the central nervous system using stereotaxic delivery. In a preferred embodiment, the vector is delivered to a desired region of a brain. It is further advantageous to deliver the vector within a region of the brain that is associated with a particular disorder. In a preferred embodiment, the region of the brain is selected from the group consisting of hypothalamus, ventromedial nucleus, and arcuate nucleus.

Another aspect of the instant invention pertains to the treatment of obesity by identifying a target site in a brain of a patient that requires modification, and transfecting at least one cell at the target site with a vector expressing a therapeutic protein, and followed by the expressing the therapeutic protein in an amount effective for modulating metabolism in the patient.

In one preferred embodiment, the target site of the brain is at least one of a hypothalamus, a ventromedial nucleus, and an arcuate nucleus.

In yet another aspect, the therapeutic protein is selected from the group consisting of brain derived neurotrophic factor (BDNF), Hip2, PGC1-alpha, estrogen receptor-alpha (ERα), glial neurotrophic factor (GNF), EPO, G-CSF, TPO, GH, IL-2, interferon-alpha receptor, interferon-beta receptor, and insulin. Alternatively, the expressed therapeutic protein is the estrogen receptor-alpha (ERα).

Accordingly, the invention relates to the treatment of obesity, by altering the basal metabolic rate in an obese subject so as to cause a reduction in body weight using the expressed therapeutic protein.

In yet another embodiment of the invention comprises administering the vector by at least one of an oral administration, a nasal administration, a bucal administration, an intravenous injection, an intra-peritoneal injection, an intrathecal administration, and a route appropriate for delivering the vector to a particular region of the brain.

In still another embodiment of this invention, a pharmaceutical composition for treating a metabolic disorder is disclosed. The pharmaceutical composition can comprise an effective amount of an adeno-associated viral vector encoding at least a portion of a gene to increase or decrease expression of a therapeutic protein in a desired region of a brain and a pharmaceutically acceptable carrier.

One embodiment of the pharmaceutical composition is used to treat metabolic disorders such as obesity, hypertension, diabetes, and atherogenic dyslipidemia. In a more preferred embodiment the disorder being treated is obesity.

In another embodiment of the pharmaceutical composition, the vector encoding at least a portion of the gene comprises an estrogen receptor-alpha (ERα) gene. The vector encoding at least a portion of the gene can also comprise a polynucleotide sequence that functions as at least one of a shRNA, a siRNA and a RNAi to decrease expression of the therapeutic protein to therapeutically effective levels. Preferably, the polynucleotide sequence comprises an estrogen receptor-alpha (ERα) gene.

In one embodiment, the gene incorporated within the AAV vector can encode therapeutic proteins such as Hip2, brain derived neurotropic factor, PGC1-α, and estrogen receptor-alpha (ERα). Preferably, the therapeutic protein can be estrogen receptor-alpha (ERα).

In still another embodiment the pharmaceutical composition, the vector is able to increase or decrease expression of the therapeutic protein in the desired region of the brain, where the region of the brain is at least one of a hypothalamus, a ventromedial nucleus, and an arcuate nucleus.

### BRIEF DESCRIPTION OF THE FIGURES

Embodiments of the invention can be more readily understood with reference to the following figures:

FIG. 1A reproduces a micrograph depicting expression of Hip2 in murine hypothalamus. Hip2 is widely expressed in the hypothalamus, with highest levels observed in the dorso-medial nucleus (DMN), the arcuate nucleus (ARC) and the ventro-medial nucleus (VMN).

FIG. 1B reproduces a micrograph depicting an assay for the detection of specific Hip2 small hairpin RNA. Suppression of Hip2 expression in brain can be performed using AAV vectors encoding for Hip2-specific small hairpin RNA (shRNA). Three different Hip2 shRNA target sequences were tested for their ability to silence the expression of the gene that codes for Hip2.

FIG. 2A. Effect of Hip2 silencing on body weight. Male mice were injected into the VMN with AAV vectors encoding for luciferase (Luc) or Hip2 shRNA. Animals from the two groups had similar body weights prior to surgery (week 0). However, mice with suppressed Hip2 levels in the VMN showed an increase in body weight shortly after vector injection.

FIG. 2B. Effect of Hip2 silencing on change in body weight of male mice. Graph of change in body weight of male mice as a function of time in weeks post-administration of AAV vectors encoding for luciferase (Luc) or Hip2 shRNA.

FIG. 2C. Hip2 silencing increases the weight of gonadal fat pads in male mice. Graph of the change in the weight of gonadal fat pads of male mice after administration of AAV vectors encoding for luciferase (Luc) or Hip2 shRNA.

FIG. 3. Effect of Hip2 silencing on cold-induced hypothermia. Core body temperature was measured at ambient temperature (22° C.) and following exposure to cold (4° C.) for 4 h. The test was performed during the dark phase of the daily cycle with full access to food and water. The animals injected with Hip2 shRNA vector developed a more profound hypothermia suggesting an impaired response to acute cold stress.

FIG. 4. Effect of Hip2 silencing on fasting-induced hypothermia. Animals were fasted for 24 h with full access to water and core temperature was measured 2 h after the onset of the dark phase and compared to the temperature of the same animals fed ad libitum (freely fed). The mice treated with Hip2 shRNA vector displayed reduced ability to maintain core body temperature following fast.

FIG. 5A. Effect of Hip2 silencing on diet-induced thermogenesis. Suppression of Hip2 levels in the VMN resulted in impaired thermogenic response following food consumption after a 24-h fast. The absolute values for body temperature were lower for Hip2 shRNA-treated mice.

FIG. 5B. The body temperature of fed mice injected with either Hip2sh or Luc increases by 2C when compared to mice that have received AAV vectors expressing the same gene in the fasted group.

FIG. 6. Effect of Hip2 silencing on daily food intake. No difference in daily food intake was observed between the two groups (A) at three weeks; (B) six weeks after surgery. At the time points noted, Hip2 shRNA-treated mice had already displayed a significant increase in body weight (FIG. 2). These results indicate that weight gain was not associated with increased consumption of food.

FIG. 7. Effect of Hip2 silencing on the response to 2-deoxy-D-glucose (2-DG) induced hypoglycemia. 2-DG significantly increased food intake over 4 h compared to saline, however, no difference was observed between the group receiving Luc shRNA and the group receiving Hip2 shRNA; (A) at 1 h following injection of 2-DG; and (B) at 4 h following injection of 2-DG.

FIG. 8. Mice injected with AAV vectors encoding Hip2 shRNA showed similar levels of daily physical activity as control mice administered Luciferase specific shRNA.

FIG. 9A. The effect of estrogen receptor-alpha (ERα) on the expression of genes responsible for neuronal glucose sensing. Decreases were observed in the expression of glucose kinase (GK) in the VMN region of the mouse brain and in glucose-responsive neuronal N43 cells after administration of AAV vectors encoding ERα sh-RNAi versus control vectors encoding for luciferase activity.

FIG. 9B. Decreases were observed in the expression of Kir6.2 in the VMN region of the mouse brain and in glucose-responsive neuronal N43 cells after administration of AAV vectors encoding ERα sh-RNAi versus control vectors encoding for luciferase.

FIG. 9C. Similar levels of expression were seen for the lactose transporter MCT1 in the VMN region of the mouse brain and in glucose responsive neuronal N43 cells after administration of AAV vectors encoding ERα sh-RNAi versus control vectors encoding for luciferase.

FIG. 10. Female mice were injected into the VMN with AAV vectoris targeting luciferase (siLuc) or ERα (siER1). Approximately three weeks after surgery, the animals were sacrificed and brain sections were stained for NeuN and ERα. Suppression of ERα expression by siER1 restricted to the VMN was observed.

FIG. 11. Mice were injected with indicated vectors and their body weight was monitored over a period of several weeks. At the end of the experiment, the animals were sacrificed and the accuracy of injections and the efficiency of ERα silencing were assessed using YFP and ERα immunostaining. Animals with unilateral (siER1-U) and bilateral (siER1-B) injections were analyzed separately. *p<0.05.

FIG. 12A. Three weeks after surgery animals were fasted for 24 h with full access to water, injected with glucose (2 mg/kg, i.p.) and their blood glucose concentration was monitored for 2 h. The test was performed during the dark phase of the daily cycle. Higher levels of blood glucose following the challenge were noted in siER1-treated mice. *p<0.05.

FIG. 12B. Three weeks after surgery were fasted for 24 h with full access to water, injected with glucose (2 mg/kg, i.p.) and their blood glucose concentration was monitored for 2 h. The test was performed during the dark phase of the daily cycle. Lower fasting glucose concentrations were noted in siER1-treated mice compared to control animals. *p<0.05.

FIG. 13A. Core body temperature was measured during the glucose tolerance test described above. While siER1-treated mice increased their body temperature after glucose injection, both baseline and induced values were significantly lower compared to control animals. *p<0.05.

FIG. 13B. Knockdown of ERα in the VMN reduced core body temperature in both fasted and ad libitum fed (freely fed) mice. *p<0.05.

FIG. 13C. Animals were systemically injected with 2-deoxy-D-glucose (250 mg/kg, i.p.) and their body temperature was monitored for 2 h. Mice treated with siER1 vector developed a more profound and sustained hypothermia compared to control animals (*p<0.05).

FIG. 13D. Animals injected with siER1 displayed reduced ability to maintain body temperature following an acute cold stress (4° C.). *p<0.05.

FIG. 14. Expression of several genes implicated in neuronal glucosensing was analyzed in dissected VMN regions by quantitative PCR. Suppression of ERα in the VMN significantly reduced mRNA levels of several genes including glucokinase, a pore-forming subunit of an ATP-dependent potassium channel, Kir6.2, and glucose transporter, GLUT3. *p<0.05.

FIG. 15. Glucose-responsive murine hypothalamic N43 cells were transduced with AAV vectors over-expressing ERα or YFP. Expression levels of several genes were analyzed 48 h later by quantitative PCR. *p<0.05.

### DETAILED DESCRIPTION OF THE INVENTION

Aspects of the present invention relate to gene therapy approaches for treating obesity as well as for the treatment of diseases such as type-II diabetes, atherogenic dyslipidemia, and coronary disease associated with disorders in metabolism. The term “metabolic syndrome” refers to a collection of factors such as central obesity, insulin resistance, hypertension, dyslipidemia, and chronic inflammation that increase the risk of individuals to diseases associated with metabolic syndrome. Particularly susceptible individuals are those who have a poor diet and nutrition, those who lead sedentary life-styles, as well as individuals with a genetic pre-disposition to diseases associated with metabolic disorders.

In some embodiments, the therapeutic potential of Hip2, estrogen receptor alpha (ERα) and/or neurotrophic growth factors in the treatment of obesity are utilized. For instance, Hip2 is a ubiquitin conjugating enzyme expressed in the brain. Recent evidence, from experiments involving obese mice, has implicated that this protein has a crucial role in energy homeostasis and body weight regulation. For example, preliminary evidence suggests that there is a direct correlation between Hip2 levels in the hypothalamus and sensitivity to diet-induced obesity in mice. Mice injected with an AAV vector encompassing the si-RNA to the gene for Hip2, showed an increase in body weight. Interestingly, the increase in body weight was due to decrease in the basal metabolic level of these animals, rather than an increase in their food intake. It therefore appears that, physiologically, Hip2 exerts control over metabolism and appears to be essential for maintaining the normal energy balance in cells. Thus, gene therapy strategies aimed at altering the steady state concentration of Hip2 provide an attractive route for treating obesity.

In addition to gene therapy directed towards the modulation of Hip2 levels in the hypothalamus, a second aim of the instant invention is the use of gene therapy to alter proteins that are downstream to Hip2 in the signaling cascade and may be involved in energy homeostasis. Recent studies have identified and focused on the protein PGC1-α, which is a key protein implicated to play a role in mitochondrial biogenesis and respiration. Studies on transcriptional regulation of energy metabolism have suggested that PGC1-α is a co-activator of the nuclear receptor PPAR-γ that regulates fat development. Interestingly, it was found that biological control of critical metabolic processes does not occur via the changes in the amounts and activities of key transcriptional factors as was originally thought, but rather occurs via transcriptional co-activation processes. For example, it has been shown that PGC1-α, co-activates both brown fat mediated thermogenesis as well as hepatic gluconeogenesis. Furthermore, PGC1-α also initiates β-oxidation of fatty acids in the liver. Thus, gene therapy approaches aimed at modulating the activity of PCP1-α could potentially find applications as therapeutics for the treatment of obesity. Alternatively, PGC1-α may pose as an attractive target in the treatment of diabetes since this protein is involved in controlling hepatic gluconeogenesis.

Growth factors, such as brain derived neurotrophic factor (BDNF), play an important role in signaling, energy metabolism, and in the overall control of body weight regulation. In particular, the ventromedial nucleus (VMN) of the hypothalamus is the key center involved in BDNF controlled energy homeostasis. In fact, there is significant support in the literature for the involvement of brain derived neurotrophic factor in body weight regulation and activity. For example, heterozygous BDNF knockout mice (Bdnf(+/−)) are hyperphagic, obese, and hyperactive; furthermore, central infusion of BDNF leads to severe, dose-dependent appetite suppression and weight loss in rats, (Friedel S, et al., Mutation screen of the brain derived neurotrophic factor gene (BDNF): Identification of several genetic variants and association studies in patients with obesity, eating disorders, and attention-deficit/hyperactivity disorder, Am. J. Med. Genet. B Neuropsychiatr. Genet. vol. 132, pg. 96, 2005). Further support for the role of BDNF in feeding behavior arises from the observation that mice with reduced expression of BDNF or its high affinity receptor, TrkB, exhibit hyperphagia and obesity. Finally, there appears to be a direct correlation between the steady state concentration of BDNF in the brain in relation to the concentration of systemic leptin.

In many obese people the leptin-controlled signaling cascade responsible for decreasing food intake in response to increasing concentration of leptin is either inefficient or non-functional. One reason for this malfunction could be due to mutations in the leptin receptor, mutations in leptin itself or the inability of the hormone to reach the desired regions of the brain. Whatever the reason for the malfunctioning of this signaling process, the observation, that an increase in systemic leptin leads to increases in BDNF expression in the brain, provides motivation for gene therapy targeting concentrations of central BDNF, and provides an attractive strategy for the treatment of leptin-resistant obesity. Thus, the invention provides methods and compositions for gene therapy aimed at treating feeding disorders and obesity by increasing the amount of BDNF centrally, or by modulating the concentration of its high affinity receptor.

Another protein that has been implicated in regulating food intake and energy expenditure is the estrogen receptor-alpha (ERα). For example, ERα knock-out mice have been shown to develop several hallmark features associated with obesity, including increased visceral adiposity, elevated insulin levels and a reduced glucose tolerance. Several studies have indicated that estrogen receptor-alpha (ERα) in the ventromedial nucleus (VMN) region of the hypothalamus is important for controlling body weight and energy homeostasis in the presence of estrogens (Musatov et al., Silencing of estrogen receptor alpha in the ventromedial nucleus of hypothalamus leads to metabolic syndrome, PNAS, vol. 104, pg. 2501, 2007). Musatov et al., have shown that si-RNA's designed to silence ERα, when delivered to the VMN of mice using adeno-associated virus (AAV) vectors cause significant gain in the body weights of these animals versus control mice injected with AAV vectors encompassing luciferase. Furthermore, these researchers were able to show that the increase in body weight was not due to an increase in food intake by the animals. Rather, they concluded that the resultant increase in body weights was most likely due to a decrease in the basal metabolic activity of the animals.

Thus, some aspects of the current application are directed towards the use of vectors such as AAV vectors to deliver the gene for ERα into the VMN neurons, so as to improve the ability of these neurons to sense blood glucose levels and thus modulate energy homeostasis. Recent studies have indicated that ERα plays a role in glucose sensing by modulation of the glucose-responsive (GR) neurons in the VMN. GR neurons use protein glucose kinase (GK) as a key regulator for sensing glucose levels. GK modulates potassium (K)-ATP channel activity via a control over the production of ATP during glycolysis. Studies using si-RNA to silence ERα have shown that ERα silencing is accompanied by a concomitant decrease in the expression of glucose kinase and a second protein Kir6.2 which is a subunit of the K-ATP channel. These results suggest a role for ERα in determining glucose levels in the hypothalamus. Furthermore, the results suggest that a method for treating obesity and other diseases linked to metabolic syndromes would be to deliver a gene capable of expressing ERα to the VMN neurons, so as to increase the in-vivo steady state level of this receptor as well as the sensitivity of the cells in the brain to changes in glucose concentrations. The current invention discloses methods and compositions for delivering the gene for ERα using AAV vectors or non-viral delivery methods.

Some aspects of the present invention employ, conventional techniques of virology, microbiology, molecular biology, and recombinant DNA techniques within the skill of the art. Such techniques are fully explained in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Thessen, ed.); Fundamental Virology, 2nd Edition, Vol. I & II (B. N. Fields and D. M. Knipe, eds.)).

### I. Definitions

So that the present application is more clearly understood, the following terms are defined. Various terms can also/alternatively, when appropriate, be delineated as typically understood by one skilled in the art.

The term “central nervous system” or “CNS” pertains to the brain, cranial nerves and spinal cord. The CNS also comprises the cerebrospinal fluid, which fills the ventricles of the brain and the central canal of the spinal cord.

As used herein, the term “polypeptide” refers to a single amino acid or a polymer of amino acid residues. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, the term “specifically binding,” refers to the interaction between binding pairs (e.g., an antibody and an antigen). In various instances, specifically binding can be embodied by an affinity constant of at most 10⁻⁶ moles/liter, at most 10⁻⁷ moles/liter, or at most 10⁻⁸ moles/liter.

As used herein, the terms “RNAi,” “siRNA,” “shRNA,” refer to a RNA polynucleotides, small interfering RNA or short hairpin RNA, respectively. RNAi, siRNA and shRNA are used in various methods of RNA interference for gene silencing, as described in more detail below.

### II. Metabolic Disorders

### Obesity

Obesity is a disease that affects many Americans. The number of overweight and obese Americans has continued to increase since 1960, a trend that is not slowing down. Today, 64.5 percent of adult Americans (about 127 million) are categorized as being overweight or obese. Each year, obesity causes at least 300,000 excess deaths in the U.S., and healthcare costs of American adults with obesity amount to approximately $100 billion.

Obesity is a chronic disease with a strong familial component. Obesity increases one's risk of developing conditions such as high blood pressure, diabetes (type 2), heart disease, stroke, gallbladder disease and cancer of the breast, prostate and colon. The tendency toward obesity is fostered by our environment: lack of physical activity combined with high-calorie, low-cost foods.

Obesity is manifested by an excess of fat mass in proportion to body size. Today, every third American is considered over-weight (Body Mass Index (BMI)>25 kg/m²), thus prompting the United States Centers for Disease Control and Prevention (CDC) to declare that obesity is reaching epidemic proportions (Cummings and Schwartz, Annu. Rev. Med. 54:453-471 ((2003)).

Obesity is caused by both genetic and environmental factors. To develop treatments for obesity, studies delineating the pathophysiology of body weight regulation are vital. It has been found that fat serves not only as a reservoir for energy but also causes the secretion of substances involved in energy homeostasis. One such substance is “leptin” a hormone whose concentration in blood serum is related to the proportion of body fat. Leptin regulates body fat content and energy expenditure by influencing the brain. Mutations in the gene for leptin or its receptors have been identified as one possible cause for obesity.

Obesity is currently treated, with only limited success, by several different strategies. These strategies primarily involve “life-style” changes (e.g. diet and exercise), small molecule based pharmaceutical therapies or surgical removal of a portion of the stomach (gastric by-pass surgery). In addition, drug treatments for obesity have been disappointing since most drugs used for treating obesity are associated with undesirable side effects that contribute to the termination of their use. Therefore, health care professionals continue to be reluctant to use pharmacotherapy in the management of obesity. Complimentary approaches to pharmacotherapy are therefore of great interest to the public.

### Diabetes

Type 2 diabetes is the most common form of diabetes. In type 2 diabetes, either the body does not produce enough insulin or the cells ignore the insulin. Insulin is necessary for the body to be able to use glucose. Cells use glucose as energy, and insulin is responsible for allowing glucose to enter cells. People with impaired metabolism have an increased propensity to develop type 2 diabetes. Diabetes has been linked to the metabolic syndrome. Particularly, recent studies have shown that ERα in the VMN region of the hypothalamus plays an important role in glucose sensing as well as in energy homeostasis. It has been shown that an under expression of the ERα protein or its absence results in obesity and an increase in body weight. Thus, delivery of viral vectors encompassing a gene capable of expressing ERα in the VMN region could provide an alternative approach to diabetes therapy.

### III. Gene Silencing through RNA Interference

RNA interference (RNAi) is a mechanism that inhibits gene expression at the stage of translation or by hindering the transcription of specific genes. Small interfering RNA strands (siRNA) are key to the RNAi process, and have complementary nucleotide sequences to the targeted RNA strand. Small hairpin RNA or short hairpin RNA (shRNA) are similar to siRNA in that the sequences are complementary to the nucleotide sequences of the targeted RNA strand, however the RNA makes a tight hairpin turn that can be used to silence gene expression via RNA interference. The shRNA hairpin structure is cleaved by the cellular machinery into siRNA which is then bound to the RNA-induced silencing complex (RISC). This complex, in turn, binds to and cleaves mRNAs which match the siRNA breaking the mRNAs down into smaller portions that can no longer be translated into protein, allowing for robust gene specific suppression.

Methods for use of RNAi, including siRNA and shRNA, and construction of RNAi vectors are known by those skilled in the art and are discussed by Engelke and Rossi in Methods in Enzymology, Volume 392: RNA Interference, 1^{st} Edition, Elsevier Academic Press, San Diego, 2005, which is herein incorporated by reference in its entirety.

### IV. Vectors

Some embodiments of the present invention utilize vectors that can be delivered to the cells of the central nervous system by using viral vectors or by using non-viral vectors. Preferred embodiments of the invention can use adeno-associated viral vectors comprising a nucleotide sequence encoding a chimeric receptor for gene delivery. AAV vectors can be constructed using known techniques to provide at least the operatively linked components of control elements including a transcriptional initiation region, an exogenous nucleic acid molecule, a transcriptional termination region and at least one post-transcriptional regulatory sequence. The control elements of the vector can be selected to be functional in the targeted cell. The resulting construct which contains the operatively linked components can be flanked at the 5′ and 3′ region with functional AAV inverted terminal repeats (ITR) sequences.

The nucleotide sequences of AAV ITR regions are known. The ITR sequences for AAV-2 are described, for example by Kotin et al. (1994) Human Gene Therapy 5:793-801; Berns “Parvoviridae and their Replication” in Fundamental Virology, 2nd Edition, Raven Press, N.Y., 1990. The skilled artisan will appreciate that AAV ITR's can be modified using standard molecular biology techniques. Accordingly, AAV ITRs used in the vectors of the invention need not have a wild-type nucleotide sequence, and can be altered, e.g., by insertion, deletion or substitution of nucleotides. Additionally, AAV ITRs can be derived from any of several AAV serotypes, including but not limited to, AAV-1, AAV-2, AAV-3, AAV-4, AAV-5, AAV-6, AAVX7, AAV-8 and the like. Furthermore, 5′ and 3′ ITRs which flank a selected nucleotide sequence in an AAV expression vector need not be identical or derived from the same AAV serotype or isolate, so long as the ITR's function as intended, i.e., to allow for excision and replication of the nucleotide sequence of interest when AAV rep gene products are present in the cell.

The skilled artisan can appreciate that regulatory sequences can often be provided from commonly used promoters derived from viruses such as: polyoma, Adenovirus 2, cytomegalovirus and Simian Virus 40. Use of viral regulatory elements to direct expression of the protein can allow for high level constitutive expression of the protein in a variety of host cells. Ubiquitously expressing promoters can also be used and include, for example, the early cytomegalovirus promoter as described by Boshart et al., Cell, vol. 41, pg. 521, 1985; herpesvirus thymidine kinase (HSV-TK) promoter as described by McKnight et al., Cell, vol. 37, pg. 253, 1984; β-actin promoters, e.g., the human β-actin promoter as described by Ng et al., Mol Cell Biol., vol. 5, pg. 2720, 1985; and colony stimulating factor-1 (CSF-1) promoter and described by Ladner et al., EMBO J., vol. 6, pg. 2693, 1987.

The AAV vector harboring the nucleotide sequence encoding a protein of interest, e.g., chimeric growth factor receptor, and a post-transcriptional regulatory sequence (PRE) flanked by AAV ITRs, can be constructed by directly inserting the nucleotide sequence encoding the protein of interest and the PRE into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g., Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling et al. (1994) Gene Therapy 1:165-169; and Zhou et al. (1994) J. Exp. Med. 179:1867-1875).

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., Molecular Cloning, a laboratory manual, Cold Spring Harbor Laboratories, N.Y., 1989. Several AAV vectors are available from the American Type Culture Collection (“ATCC”).

In order to produce recombinant AAV particles, an AAV vector can be introduced into a suitable host cell using known techniques, such as by transfection. A number of transfection techniques are generally known in the art. See, e.g.; Sambrook et al., Molecular Cloning a laboratory manual, Cold Spring Harbor Laboratories, N.Y., 1989; Davis et al., Basic Methods in Molecular Biology, Elsevier, San Diego, 1986. Particularly suitable transfection methods include calcium phosphate co-precipitation (Graham et al., Virology, vol. 52, pg. 456, 1973), direct micro-injection into cultured cells (Capecchi, Cell, vol. 22, pg. 479, 1980), electroporation (Shigekawa et al., BioTechniques, vol. 6, pg. 742, 1988), liposome mediated gene transfer (Mannino et al., BioTechniques, vol. 6, pg. 682, 1988), lipid-mediated transduction (Felgner et al., Proc. Natl. Acad. Sci. USA, vol. 84, pg. 7413, 1987), and nucleic acid delivery using high-velocity microprojectiles (Klein et al., Nature, vol. 327, pg. 70, 1987).

Suitable host cells for producing recombinant AAV particles include, but are not limited to, microorganisms, yeast cells, insect cells, and mammalian cells, that can be, or have been, used as recipients of a exogenous nucleic acid molecule.

Host cells containing the above-described AAV vectors must be rendered capable of providing AAV helper functions in order to replicate and encapsidate the expression cassette flanked by the AAV ITRs to produce recombinant AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, AAV helper functions include one, or both of the major AAV open reading frames (ORFs), namely the rep and cap coding regions, or functional homologues thereof.

Alternatively, a vector can be a virus other than the adeno-associated virus, or portion thereof, which allows for expression of a nucleic acid molecule introduced into the viral nucleic acid. For example, replication defective retroviruses, adenoviruses, herpes simplex virus, and lentivirus can be used. Protocols for producing recombinant retroviruses and for infecting cells in vitro or in vivo with such viruses can be found described by Ausubel et al. in Current Protocols in Molecular Biology, Greene Publishing Associates, 1989, Sections 9.10-9.14 and other standard laboratory manuals. Examples of suitable retroviruses include pLJ, pZIP, pWE and pEM which are well known to those skilled in the art. Examples of suitable packaging virus lines include Crip, Cre, 2 and Am. The genome of adenovirus can be manipulated such that it encodes and expresses the protein of interest but is inactivated in terms of its ability to replicate in a normal lytic viral life cycle. See e.g., Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art.

Alternatively, the vector can be delivered using a non-viral delivery system. This includes delivery of the vector to the desired tissues in colloidal dispersion systems that include, for example, macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. In order for a liposome to be an efficient gene transfer vehicle, the following characteristics should be present: (1) encapsulation of the genetic material at high efficiency while not compromising the biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al. (1988) Biotechniques, 6:682). Examples of suitable lipid liposome production include phosphatidyl compounds, such as phosphatidylglycerol, phosphatidylcholine, phosphatidylserine, phosphatidylethanolamine, sphingolipids, cerebrosides, and gangliosides. Additional examples of lipids include, but are not limited to, polylysine, protamine, sulfate and 3b-[N—(N′,N′dimethylaminoethane) carbamoyl] cholesterol.

### V. Pharmaceutical Compositions And Pharmaceutical Administration

The vector or the synthetic dimerizer used with some embodiments of the present invention can be incorporated into pharmaceutical compositions suitable for administration to a subject. In some particular embodiments, the pharmaceutical composition comprises the vector of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector or pharmaceutical composition.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular, intrathecal). In one embodiment, the vector is administered by intravenous infusion or injection. In another embodiment, the vector is administered by intramuscular or subcutaneous injection. In another embodiment, the vector is administered perorally. In the most preferred embodiment, the vector is delivered to a specific location using stereostatic delivery.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., vector) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be achieved by including an agent in the composition that delays absorption, for example, monostearate salts and gelatin.

The vector of the present invention can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems by J. R. Robinson, ed., Marcel Dekker, Inc., New York, 1978.

The pharmaceutical compositions of the invention can include a “therapeutically effective amount” or a “prophylactically effective amount” of the vectors of the invention. A “therapeutically effective amount” refers to an amount effective, at dosages and for periods of time necessary, to achieve the desired therapeutic result. A therapeutically effective amount of the vector can vary according to factors such as the disease state, age, sex, and weight of the individual, and the ability of the vector to elicit a desired response in the individual. A therapeutically effective amount can also be one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose can be used in subjects prior to or at an earlier stage of disease, the prophylactically effective amount can be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It can be especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention can be dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Some aspects of the current invention are directed towards the use of vectors, such as AAV vectors, that can deliver at least a portion of a gene, such as the gene for ERα, to a cell such as a mammalian brain cell, either in vitro or in vivo. In some embodiments, the vectors can be delivered to a region of the brain, such as the hypothalamus, ventromedial nucleus (VMN), and arcuate nucleus. As disclosed herein, experiments have been conducted in mice where suppression of ERα by shRNAs led to an increase in body weight, decreased glucose tolerance, reduced body temperature and reduced expression of key glucosensing genes, such as glucose kinase and Kir6.2, a pore-forming subunit of the potassium ATP channel. As well, other experiments documented the overexpression of ERα in glucose-responsive hypothalamic N43 cells, which led to increased expression of glucose kinase (GK) and Kir6.2.

While not being bound by any particular theory, it is believed that the increased expression of GK can result in a desired therapeutic outcome. Cells of the ventromedial nucleus have been shown to sense blood glucose levels and thus modulate energy homeostasis. Glucose responsive neurons use protein GK as a regulator for sensing glucose levels. GK modulates potassium (K)-ATP channel activity via a control over the production of ATP during glycolysis. Increased levels of GK, as demonstrated by Niswender et al., J. Biol. Chem., vol. 272, pg. 22564, 1997; and Niswender et al., J. Biol. Chem., vol. 272, pg. 22570, 1997, herein incorporated by reference in their entirety, have been shown to result in increased glycogen synthesis, decreased body weight and increased metabolism in murine models. Accordingly, the present application supports the notion that ERα plays a significant role in determining glucose levels in the brain (e.g., hypothalamus), specifically the VMN and that increasing ERα expression levels leads to an increase in GK expression and function (as demonstrated by Kir6.2 increased expression). Overexpression of ERα in murine models demonstrated that increased GK led to increased metabolism and decreased body weight. Thus, increasing expression of ERα in the brain of a mammal, specifically the VMN, can increase GK expression and lead to a decrease in body weight.

Furthermore, the results suggest that a method for treating obesity and other diseases linked to metabolic syndromes can be delivering a gene capable of expressing ERα to the VMN neurons, so as to increase the in-vivo steady state level of this receptor as well as the sensitivity of the cells in the brain to changes in glucose concentrations. Thus, the current invention discloses methods directed to treating metabolic disorders, such as obesity, by transfecting at least one mammalian cell with a vector, such as an AAV vector, expressing a therapeutic protein, like ERα, to increase or decrease the level of the therapeutic protein, thereby treating the disorder. or non-viral delivery methods.

One skilled in the art can appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

### Effect of Hip2 Silencing on Body Weight of Mice

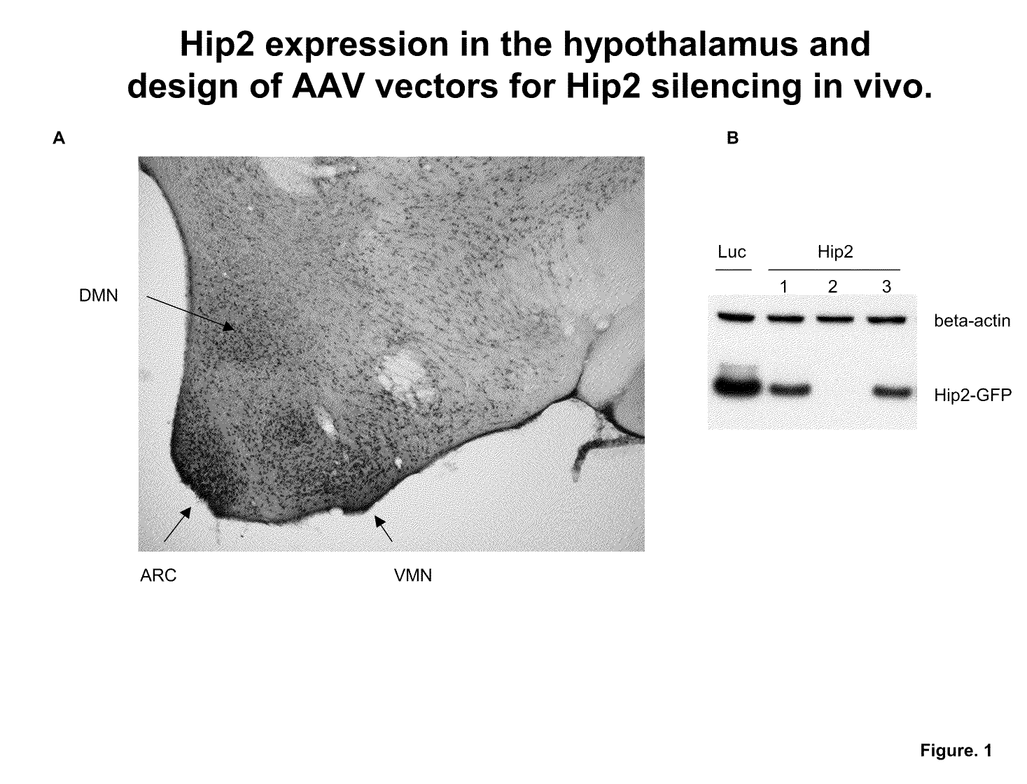

Prior to experimentation, Hip2 expression was characterized in murine hypothalamus. FIG. 1A shows the wide expression pattern of Hip2, with highest levels observed in dorso-medial nucleus (DMN), the arcuate nucleus (ARC) and the ventro-medial nucleus (VMN).

Male mice were injected with AAV vectors encoding Hip2 specific small hairpin RNA's (shRNA). AAV vectors encoding luciferase specific shRNA's were used as controls. The vectors were delivered to the ventromedial nucleus (VMN) region of the brain. After several weeks of monitoring the mice, the animals were sacrificed and the level of Hip2 suppression was assessed. Brain tissue from the mice injected with AAV vectors encoding for three different Hip2-specific shRNAs or a luciferase control were analyzed by PCR for the ability to silence Hip2 gene expression. Of the three, one shRNA was able to drastically reduce the level of Hip2 present in the brain, as measured by PCR.

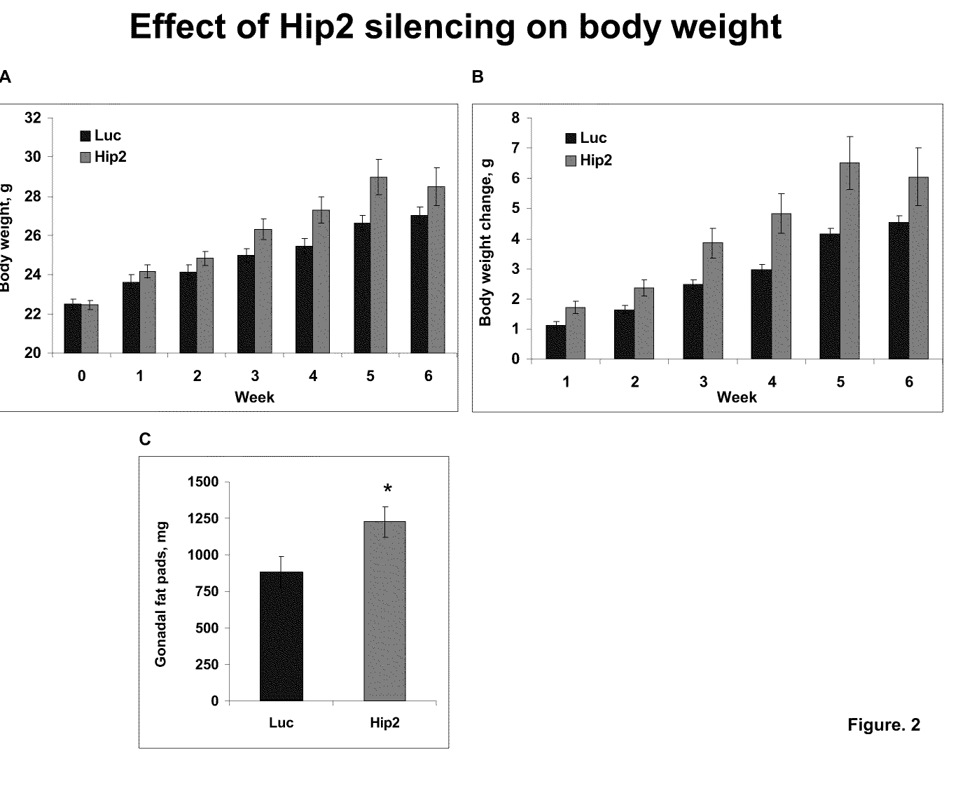

To determine the effect Hip2 silencing has on the mice, body weights were measured as an additional parameter. The body weights of mice in each group were determined prior to surgery and were found to be similar. Subsequently, the body weights of the animals in the two groups were measured each week following administration of AAV vectors for up to six weeks. The results are graphically depicted in FIG. 2A. The graph shows that the body weights of mice from the group that received an injection of AAV vectors encoding for Hip2 shRNA are greater than the body weights of mice that received injections of the control AAV vectors. The increase in the body weights of mice receiving Hip2 shRNAs was observed as early as one week post administration of the AAV vectors encoding for Hip2 shRNAs. However, the difference in body weights between the two groups was greater for weeks four, five, and six following surgery. FIG. 2B shows the overall change in body weights of mice within each group by comparing the weight of animals at weeks 1-6 post injection of the AAV vectors encoding either Hip2 shRNA or Luc shRNA, to the body weights of animals prior to surgery. Silencing of Hip2 expression resulted in a greater increase in the body weights of animals versus animals from the control group (Luc shRNA). Furthermore, it was noted that the increase in body weight resulted in increased accumulation of visceral fat, seen in FIG. 2C. The accumulation of visceral fat tissue was greater for animals in the Hip2 group than control animals.

### Example 2

### Effect of Hip2 Silencing on Cold-Induced Hypothermia

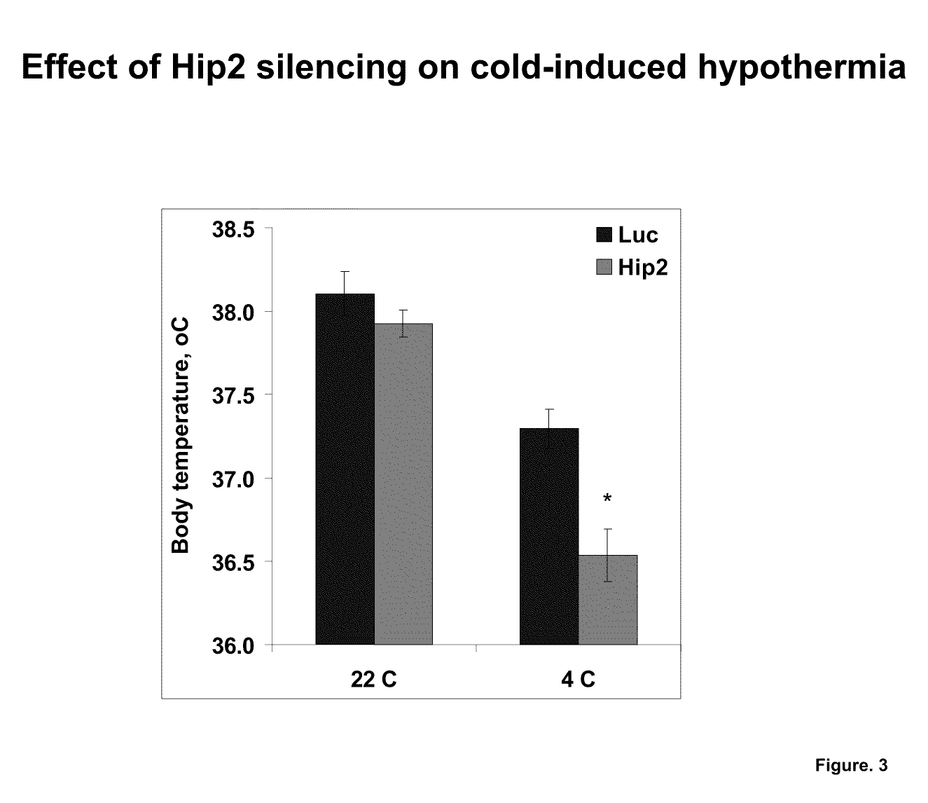

Male mice were divided into two groups, one group was injected with AAV vectors encoding for Hip2 shRNA while the other group was injected with AAV vectors encoding Luc shRNA. Mice were separated in cages according to group and placed in a room maintained at an ambient temperature of 22° C., or in cages placed in a room at 4° C. Mice were permitted access to food and water ad libitum (freely available), and measurements of core body temperature were made during the dark phase of the daily cycles. After 4 h at either 22° C., or at 4° C., the core body temperatures of the animals were recorded. The results are shown in FIG. 3. Animals maintained at 4° C. had lower core body temperatures than animals maintained at 22° C. At either temperature, the animals that had received injections of Hip2 shRNA had lower core body temperatures than animals in the control group. However, the core body temperatures of mice receiving Hip2 shRNA were much lower for animals kept at 4° C. than for animals kept at an ambient temperature of 22° C. These results indicate that Hip2 silencing impairs the animals' ability to respond to stress due to cold.

### Example 3

### Effect of Hip2 Silencing on Fasting-Induced Hypothermia

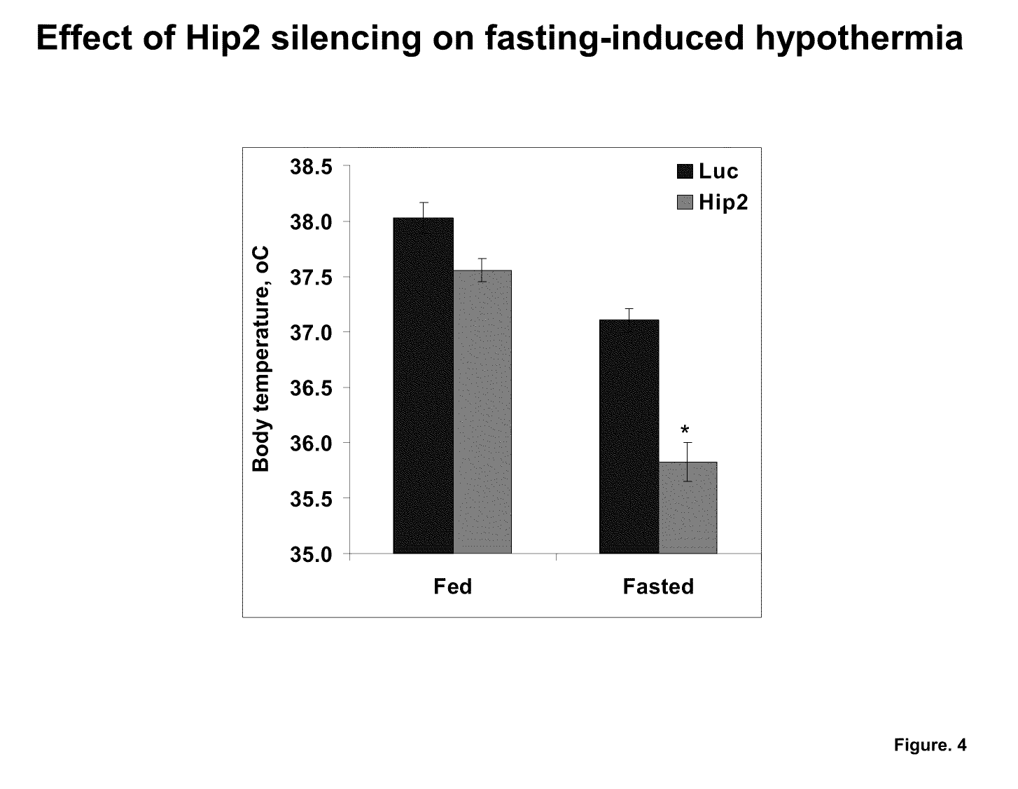

Male mice were divided into two groups, one group was injected with AAV vectors encoding for Hip2 shRNA while the other group was injected with AAV vectors encoding Luc shRNA. Mice from each group were either fasted for 24 h or allowed access to food ad libitum. For both the fasted and fed mice, access to water was allowed ad libitum. The measurements of core body temperature were made 2 h after the onset of the dark phase of the daily cycles. The results are shown in FIG. 4. The core body temperature was lower for animals that had been injected with the Hip2 shRNA versus control animals in both the fasted and fed groups. However, this effect was more pronounced for the animals that had fasted for 24 h prior to the measurements. This indicates that animals treated with Hip2 shRNA have a reduced ability to maintain core body temperature following fasting.

### Example 4

### Effect of Hip2 Silencing on Diet-Induced Thermogenesis

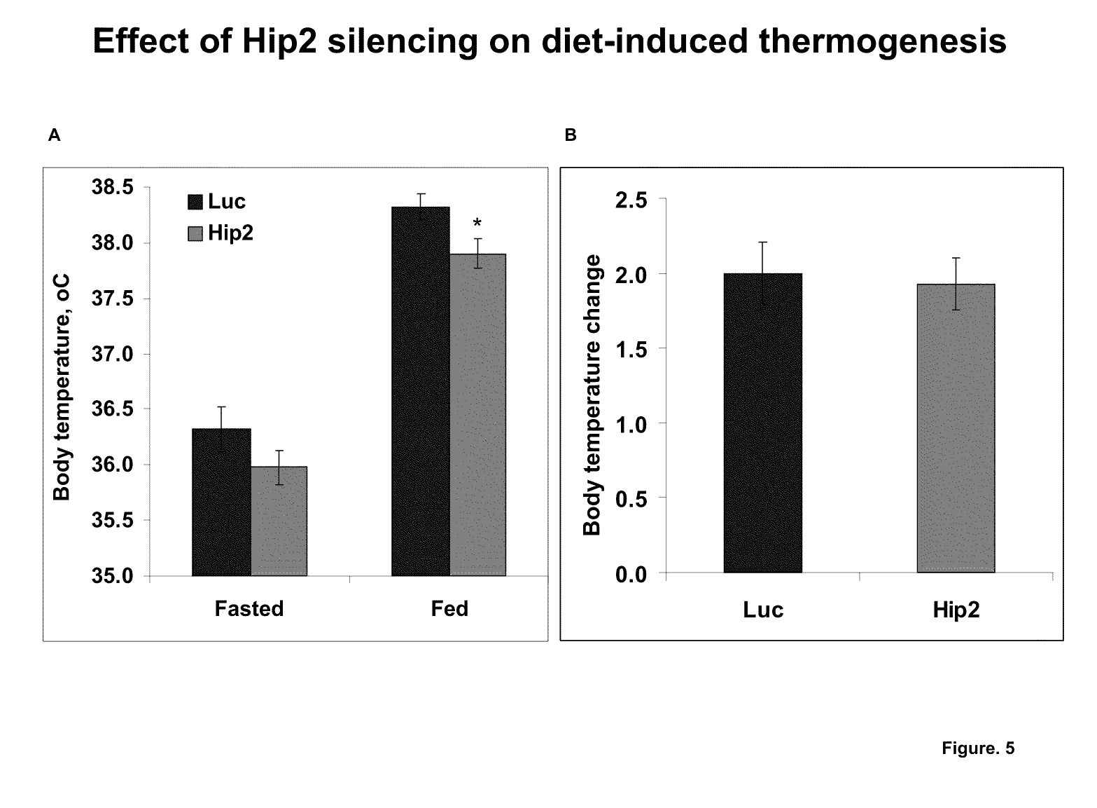

Male mice were divided into two groups, one group was injected with AAV vectors encoding for Hip2 shRNA while the other group was injected with AAV vectors encoding Luc shRNA. Mice from each group were either fasted for 24 h or allowed access to food ad libitum. Mice in both the fasted and fed groups were allowed access to water ad libitum. FIG. 5A shows that the body temperature of fasted mice was lower than the body temperature of animals that were allowed to feed ad libitum. However, the overall difference in body temperature for mice injected with Hip2 shRNA in both the fasted and fed group was approximately 2° C., similar to the overall difference in body temperatures for mice in the fed and fasted groups that received an injection of Luc shRNA.

Hip2 has been implicated to play a role in metabolism and in energy homeostasis making Hip2 an attractive target for therapeutic intervention, especially for the treatment of diseases associated with the metabolic syndrome. Results from various in vivo experiments have shown that suppression of endogenous Hip2 in mice resulted in an increase in the body weight of these animals. The increase in body weight was most often accompanied by an increase in the visceral adiposity in animals, which strongly correlated with several hallmark features of diseases associated with metabolic syndrome.

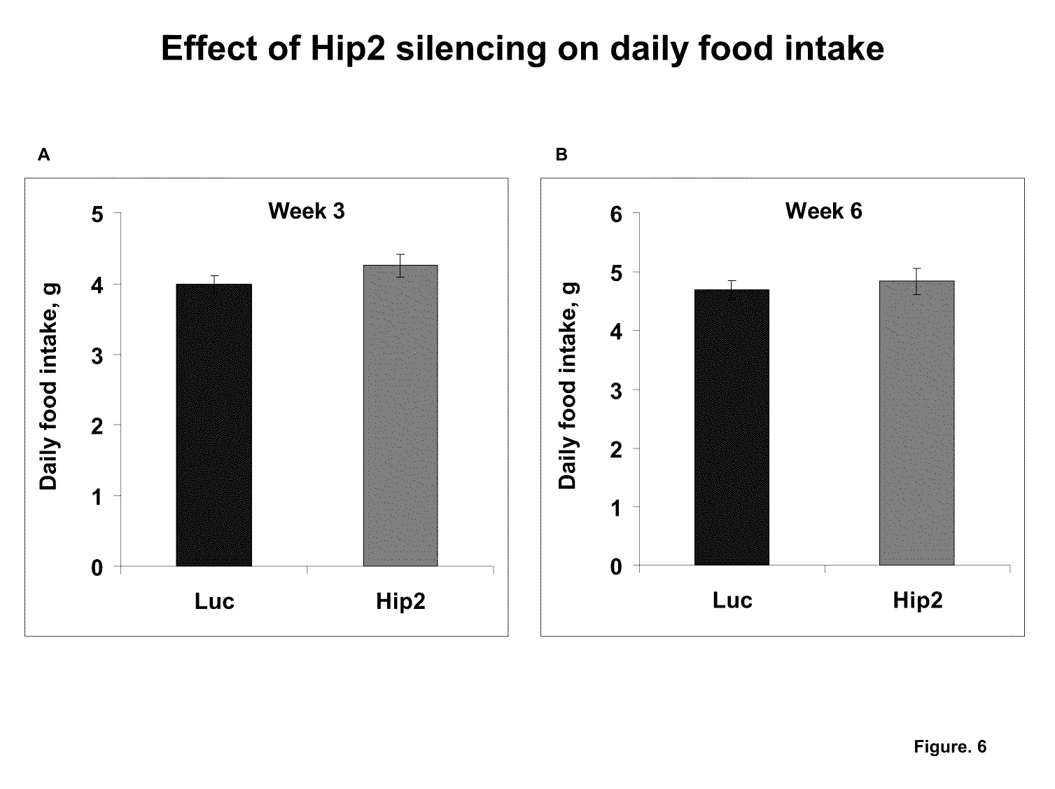

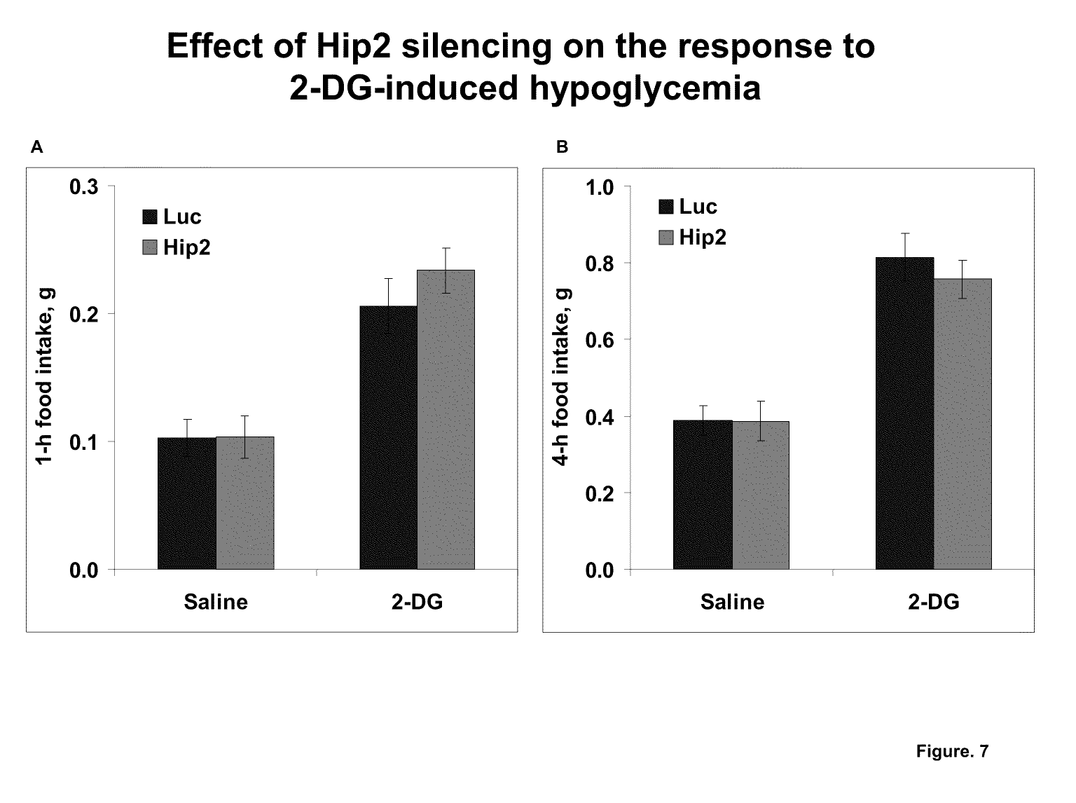

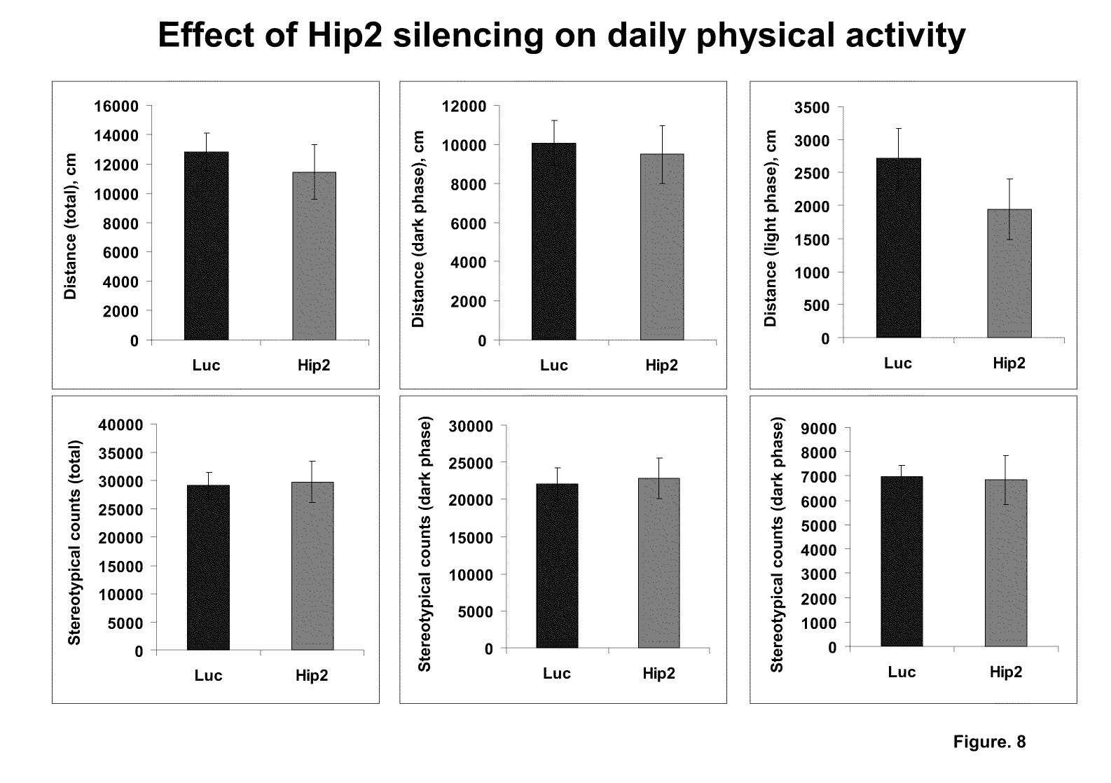

Silencing of Hip2 was achieved by AAV vectors encoding the Hip2 small hairpin RNA (Hip2 shRNA) delivered into the VMN region of the brain via injection. AAV vectors encoding the luciferase shRNA were used as controls. While reduction of Hip2 levels resulted in increased body weight as well as a more acute response in the animals ability to regulate and maintain core body temperature, Hip2 silencing did not alter the animals food intake (FIG. 6) nor did it affect their daily physical activities (FIG. 8). Furthermore, reduction of Hip2 levels also had no effect on the ability of the VMN neurons to sense glucose levels in blood. When mice were administered 2-deoxy-D-glucose (2DG) a non-metabolizable form of glucose that induces hyperphagia, there was no difference in the food intake between mice injected with the AAV vector for Hip2 shRNA versus mice injected with the vector for Luc shRNA (FIG. 7). These results show that Hip2 is not essential for the VMN neurons ability to sense blood glucose levels.

### Example 5

### Modulation of Neuronal Glucose Sensing by Estrogen Receptor Alpha (ERα)

Recent experiments have demonstrated that RNAi-mediated silencing of ERα in the neurons of the ventromedial nucleus (VMN) of the hypothalamus of adult mice leads to behavioral and physiological abnormalities closely resembling a metabolic syndrome. VMN contains populations of glucose-responsive (GR) neurons critical for the regulation of energy homeostasis by glucose. The majority of GR neurons use glucokinase (GK) as the key regulator of glucosensing. GK appears to modulate potassium (K)-ATP channel activity via its gatekeeper role in the glycolytic production of ATP. Consequently, GR neurons change their membrane potential and firing rate as a function of glucose concentration eventually initiating a counter regulatory response to elevated or reduced glucose levels in the blood.

It was hypothesized that ERα modulates the glucosensing function of GR neurons, thus integrating estrogen and glucose signaling pathways. The preliminary experiments have revealed that overexpression of ERα in a glucose-responsive murine hypothalamic cell line, N43, lead to upregulation of GK and Kir6.2, a pore-forming subunit of the K(ATP) channel. Concomitantly, the levels of glucose and lactose transporters were not affected. Furthermore, silencing of ERα in the VMN neurons of female mice suppressed the expression of both GK and Kir6.2. These results suggest an important role of ERα in glucosensing by hypothalamic neurons.

Furthermore, changes in ERα signaling in this brain region may be implicated in the development of the metabolic syndrome. Given that elevated levels of GK and Kir6.2 are expected to improve brain sensitivity to glucose, overexpression of ERα in the VMN neurons using viral vectors may thus hold promise as a novel approach to treat the metabolic syndrome in humans.

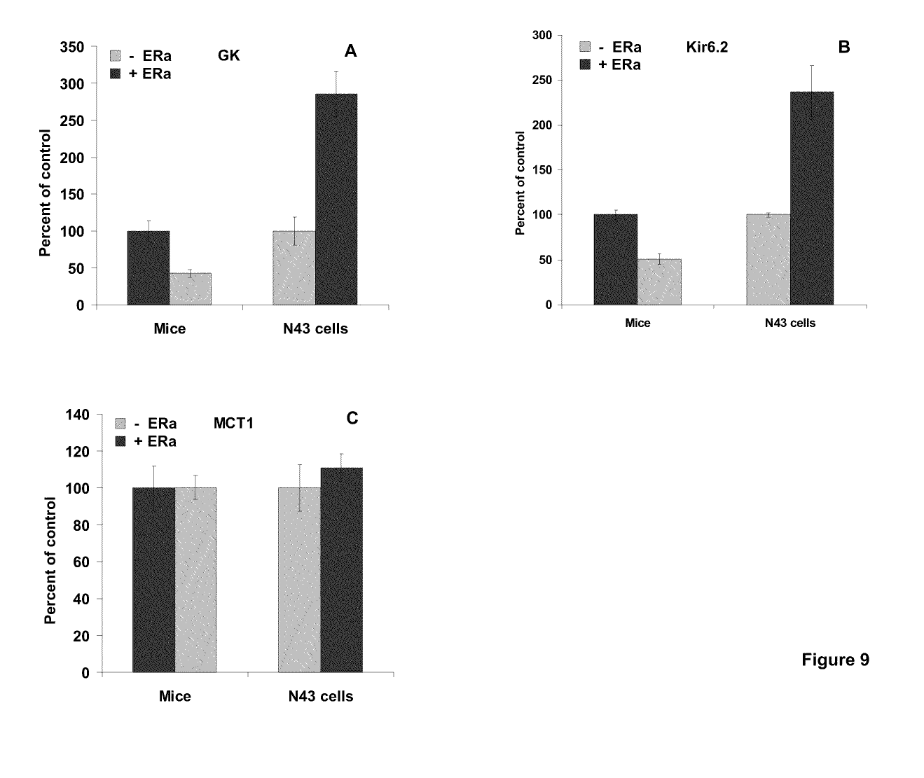

The effect ERα levels have on expression of genes essential for neuronal glucose sensing was determined. To address the role of endogenous ERα in glucose sensing by the VMN neurons, female mice were injected with AAV vectors encoding for small hairpin RNAs (shRNAs) targeting ERα or luciferase (negative control). Several weeks after surgery, brain regions containing VMN were dissected and the levels of GK, Kir6.2 and a lactose transporter MAT1 were analyzed by quantitative PCR, see FIGS. 9A, B and C respectively. Suppression of endogenous ERα resulted in downregulation of GK and Kir6.2 but not MAT1. Consistent with these results, overexpression of ERα in a murine hypothalamic N43 cell line using AAV vectors induced both GK and Kir6.2.

### Example 6

### AAV-Mediated Estrogen Receptor Alpha (ERα) Silencing in the VMA

AAV vectors were designed to express small hairpin RNA (shRNA) for silencing gene expression targeting murine ERα (GGCATGGAGCATCTCTACA, SEQ ID NO.: 1) or firefly luciferase (CCGCTGGAGAGCAACTGCAT, SEQ ID NO.:2) under the control of the human Hi promoter. In addition, both vectors contained a second expression cassette for destabilized yellow fluorescent protein (YFP). The latter was used as a reporter to visualize transduced neurons. Vector stocks were generated using a helper-free AAV-2 plasmid transfection system, purified by heparin affinity chromatography and dialyzed against PBS. AAV genomic titers were determined by quantitative PCR and adjusted to 10⁹ particles per ml.

Stereotaxic surgery was performed to administer AAV vectors directly to the ventromedial nucleus (VMN) of the hypothalamus of adult mice and determined the effect ERα gene silencing had on tissues from the mice. All stereotaxic surgical procedures were performed on gonad-intact female mice (8-12 weeks old) under ketamine/xylazine anesthesia. Vectors (1 μl, 109 particles) were injected into the VMN (AP−0.9, ML+/−0.6, DV−5.8) bilaterally over 10 min using a 10-ml Hamilton syringe and an infusion pump (World Precision Instruments). The needle was left inserted for an additional 5 min and then slowly withdrawn. Animals which were successfully targeted bilaterally and those with unilateral injections were analyzed as separate groups.

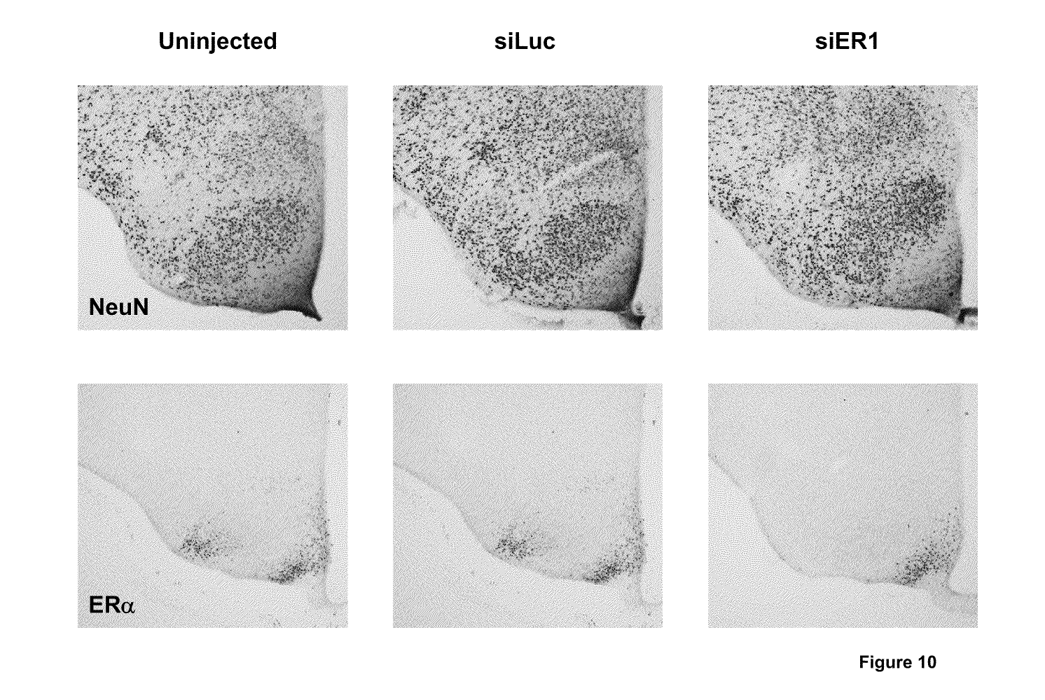

Approximately three weeks after surgery, female mice that were injected into the VMN with AAV vectors targeting luciferase (siLuc) or ERα (siER1) were sacrificed and perfused with 4% paraformaldehyde. The brains were analyzed by immunohistochemistry using a free floating section method and the following primary antibodies and stained for NeuN and ERα using anti-GFP (Abcam, ab290, 1:10,000), anti-ERα (Upstate Biotechnology, C1355, 1:10,000), and anti-NeuN (Chemicon, MAB377, 1:10,000). Accuracy of injections and the efficiency of ERα silencing were also assessed with YPF and ERα immunostaining. Animals with unilateral (siER1-U) and bilateral (siER1-B) injections were analyzed separately, p<0.05. It was noted that suppression of ERα expression by siER1 was restricted to the VMN, as shown in FIG. 10.

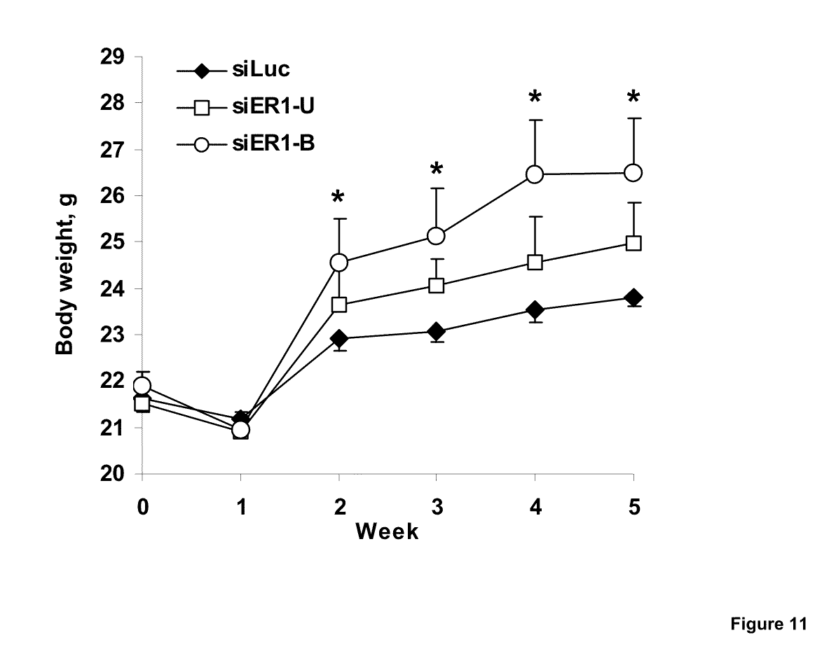

Prior to sacrificing the animals, the body weights of the mice injected with the indicated vectors were monitored over a period of 3 to 5 weeks post surgery. Statistically significant increases (p<0.05) in body weight were observed in siER1-B and siER1-U injected mice as opposed to marginal increases in body weight as seen in siLuc injected mice (FIG. 11).

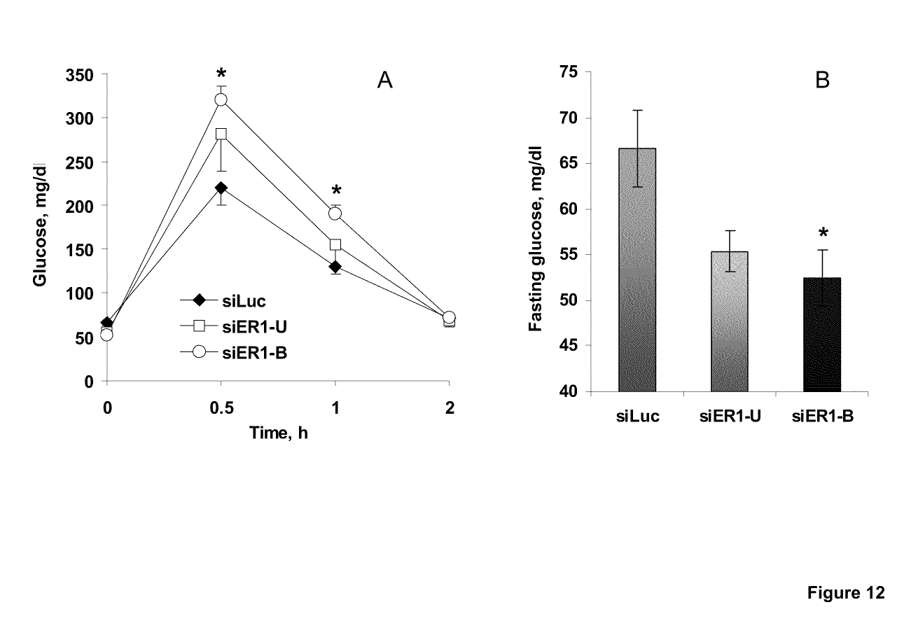

The effect ERα silencing had on fasting glucose levels was also investigated. Three weeks after surgery, glucose starved animals (performed during the dark phase of the daily cycle) were injected with glucose (2 mg/kg, i.p.) and monitored their blood glucose concentration for 2 h. Prior to the challenge, siER1-treated mice exhibited lower fasting glucose concentrations when compared to control animals (FIG. 12A). However after glucose challenge, higher levels of blood glucose in siER1-treated mice (p<0.05) (FIG. 12B) than controls were noted, indicating reduced glucose tolerance when ERα is silenced.

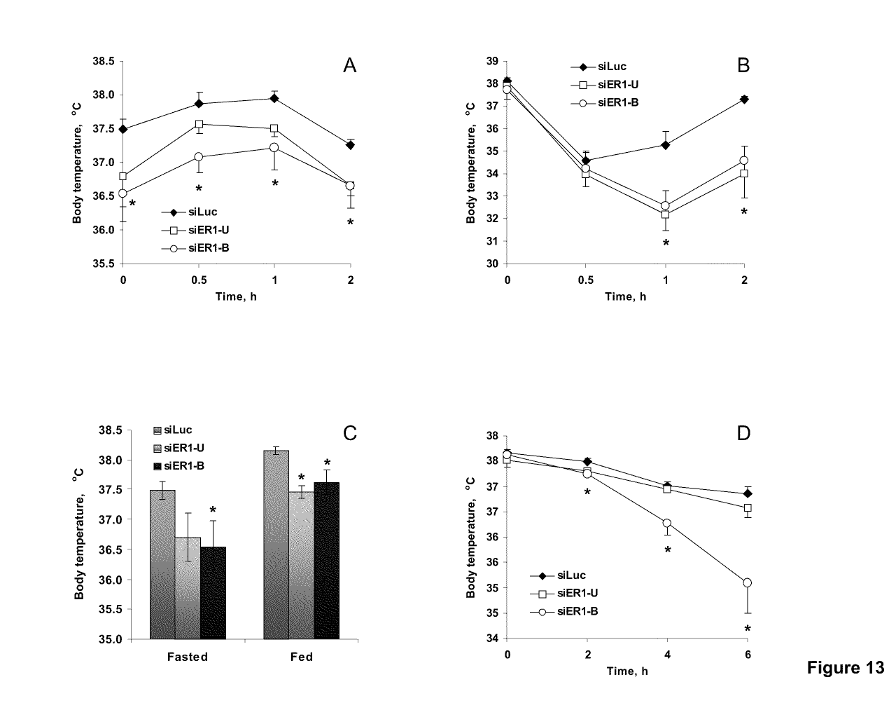

Core body temperature as an additional parameter during the glucose tolerance test described above was also measured. siER1-treated mice increased their body temperature after glucose injection. However, FIG. 13A illustrates that both baseline values and after glucose challenge values were significantly lower for siER1-treated mice when compared to control animals (p<0.05). Similar reductions in body temperature were also noted in siER1-treated mice when fasted and ad libitum fed (freely available), shown in FIG. 13B.

In another set of experiments, the effects of glucopenia and acute temperature stress had on body temperature was measured. Animals were systemically injected with 250 mg/kg of 2-deoxy-D-glucose (which is incapable of undergoing glycolysis) intraperitoneally and their body temperature was monitored for 2 h. Mice treated with siER1 vector developed a more profound and sustained hypothermia apparent in as little as 30 mins after induced glucopenia as compared to control animals, illustrated in FIG. 13C. Similarly in FIG. 13D, when mice were exposed to acute cold stress (4° C.), animals injected with siER1 displayed reduced ability to maintain body temperature.

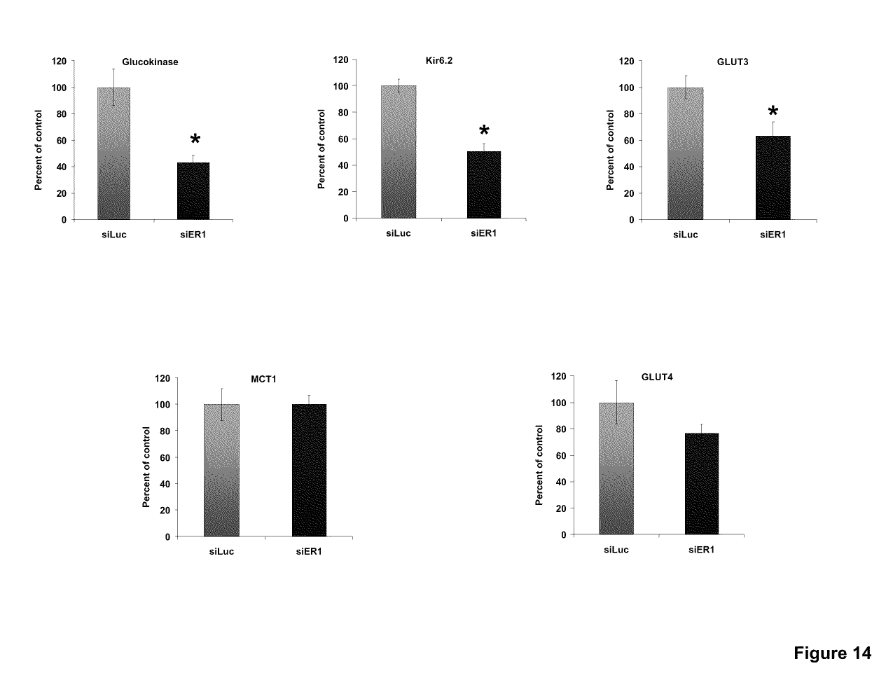

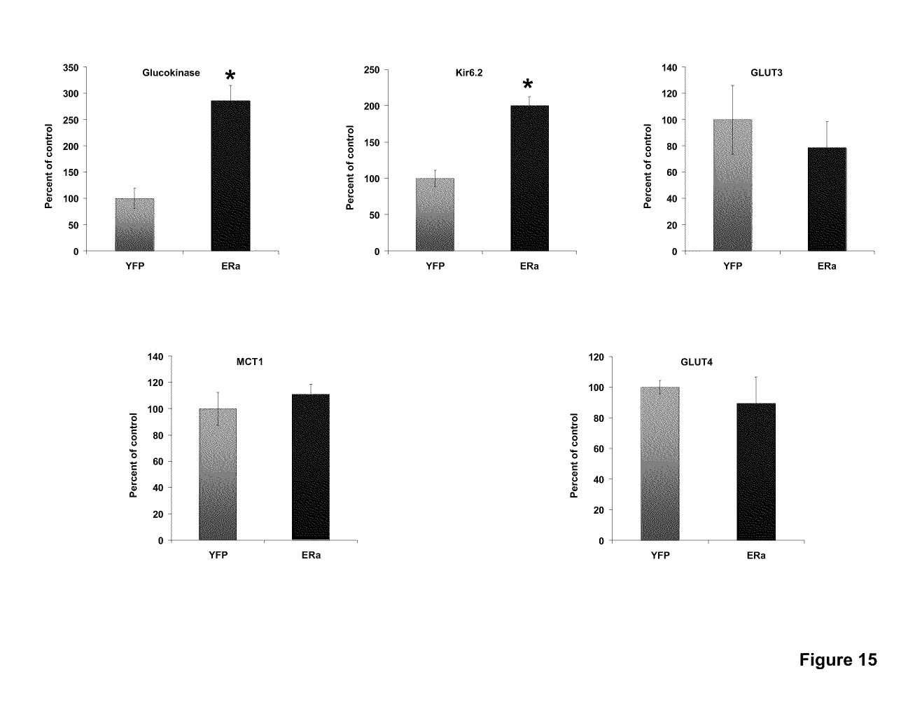

To better understand the role ERα plays in glucose metabolism, tissues from dissected VMN regions of injected mice were analyzed for expression of several genes implicated in neuronal glucosensing by quantitative PCR. Suppression of ERα in the VMA resulted in the significant reduction (p<0.05) in mRNA levels of several genes including glucokinase (an enzyme that increases metabolism of glucose through phosphorylation), Kir6.2 (a pore-forming subunit of the K(ATP) channel) and glucose transporter GLUT3, as shown in FIG. 14.

### Example 7

### AAV-Mediated Estrogen Receptor Alpha (ERα) Overexpression in Glucose-Responsive Cells In Vitro

To determine if overexpression of ERα has the opposite effect as ERα silencing, ERα in a glucose sensitive cell line was overexpressed. Glucose-responsive murine hypothalamic N43 cells were transduced with over-expression ERα or YFP AAV vectors. Expression levels of glucosensing genes were analyzed 48 h later by quantitative PCR and significant expression increases (p<0.05) were observed for glucokinase and Kir6.2.

While the present invention has been described in terms of specific methods, and compositions, it is understood that variations and modifications will occur to those skilled in the art upon consideration of the present invention. For example, the methods and compositions discussed herein can be utilized beyond treating metabolic disorders and pharmaceutical compositions in some embodiments. As well, the features illustrated or described in connection with one embodiment can be combined with the features of other embodiments. Such modifications and variations are intended to be included within the scope of the present invention. Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims.

All publications and references are herein expressly incorporated by reference in their entirety. The terms “a” and “an” can be used interchangeably, and are equivalent to the phrase “one or more” as utilized in the present application. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed:

1. A method for treating a metabolic disorder comprising:
identifying the metabolic disorder;
providing at least a portion of a gene to increase or decrease expression of a therapeutic protein;
incorporating the gene into a vector;
transfecting the vector into at least one cell in a central nervous system; and
increasing or decreasing expression of the therapeutic protein in at least one cell to thereby treat the metabolic disorder.

2. The method of claim 1, wherein the metabolic disorder is selected from the group consisting of obesity, type-2 diabetes, hypertension, and atherogenic dyslipidemia.

3. The method of claim 1, wherein the step of providing the gene comprises providing a polynucleotide sequence that functions as at least one of a shRNA, a siRNA, and a RNAi.

4. The method of claim 3, wherein the polynucleotide comprises an estrogen receptor-alpha (ERα) gene.

5. The method of claim 1, wherein the therapeutic protein is selected from the group consisting of Hip2, PGC1-alpha, and estrogen receptor-alpha (ERα).

6. The method of claim 1, wherein the therapeutic protein is an estrogen receptor-alpha (ERα).

7. The method of claim 1, wherein the gene is at least a portion of a gene from the group consisting of Hip2, PGC1-alpha, and estrogen receptor-alpha (ERα).

8. The method of claim 7, wherein the gene is at least a portion of an ERα gene.

9. The method of claim 1, wherein the step of incorporating the gene into the vector comprises incorporating the gene into a viral vector.

10. The method of claim 9, wherein the vector is selected from the group consisting of adeno-associated viral vector, herpes viral vector, parvoviral vector, and lentiviral vector.

11. The method of claim 9, wherein the viral vector is an adeno-associated viral vector (AAV).

12. The method of claim 11, wherein the AAV is a recombinant AAV having a cap-region from AAV type (1) and a rep-region from AAV type (2).

13. The method of claim 1, wherein the step of incorporating the gene into the vector comprises incorporating the gene into a non-viral vector.

14. The method of claim 13, wherein the non-viral vector is a liposome-mediated delivery vector.

15. The method of claim 1, wherein the step of transfecting the vector comprises delivering the vector to a desired region of the central nervous system using stereotaxic delivery.

16. The method of claim 1, wherein the step of transfecting the vector comprises delivering the vector to a desired region of a brain.

17. The method of claim 16, wherein the region of the brain is selected from the group consisting of hypothalamus, ventromedial nucleus, and arcuate nucleus.

18. A method for treating obesity comprising:
identifying a target site in a brain for modification of a patient in need thereof;
transfecting at least one cell at the target site with a vector expressing a therapeutic protein,
expressing the therapeutic protein in an amount effective for modulating metabolism in the patient.

19. The method of claim 18, wherein the target site of the brain is at least one of a hypothalamus, a ventromedial nucleus and an arcuate nucleus.

20. The method of claim 18, wherein the therapeutic protein is selected from a group consisting of brain derived neurotrophic factor (BDNF), Hip2, PGC1-alpha, estrogen receptor-alpha (ERα), glial neurotrophic factor (GNF), EPO, G-CSF, TPO, GH, IL-2, interferon-alpha receptor, interferon-beta receptor, and insulin receptor.

21. The method of claim 18, wherein the therapeutic protein is the estrogen receptor-alpha (ERα).

22. The method of claim 18, wherein the step of expressing the therapeutic protein comprises altering a basal metabolic rate to cause a reduction in body weight.

23. The method of claim 18, wherein the step of transfecting at least one cell comprises administering the vector by at least one of an oral administration, a nasal administration, a bucal administration, an intravenous injection, an intra-peritoneal injection, an intrathecal administration, and a route appropriate for delivering the vector to a particular region of the brain.

24. A pharmaceutical composition for treating a metabolic disorder comprising:
a effective amount of an adeno-associated viral vector encoding at least a portion of a gene to increase or decrease expression of a therapeutic protein in a desired region of a brain; and
a pharmaceutically acceptable carrier.

25. The pharmaceutical composition of claim 24, wherein the disorder is selected from the group consisting of obesity, hypertension, diabetes, and atherogenic dyslipidemia.

26. The pharmaceutical composition of claim 25, wherein the disorder is obesity.

27. The pharmaceutical composition of claim 24, wherein the at least the portion of the gene comprises an estrogen receptor-alpha (ERα) gene.

28. The pharmaceutical composition of claim 24, wherein the gene comprises a polynucleotide sequence that functions as at least one of a shRNA, a siRNA and a RNAi to decrease expression of the therapeutic protein to therapeutically effective levels.

29. The pharmaceutical composition of claim 28, wherein the polynucleotide sequence comprises an estrogen receptor-alpha (ERα) gene.

30. The pharmaceutical composition of claim 24, wherein the therapeutic protein is selected from the group consisting of Hip2, brain derived neurotropic factor, PGC1-α, and estrogen receptor-alpha (ERα).

31. The pharmaceutical composition of claim 24, wherein the therapeutic protein is estrogen receptor-alpha (ERα).

32. The pharmaceutical composition of claim 24, wherein the region of the brain is at least one of a hypothalamus, a ventromedial nucleus, and an arcuate nucleus.


SEQUENCE LISTING (2 sequences)

SEQ ID NO: 1 - DNA, 19 bp, Mus musculus
ggcatggagcatctctaca

SEQ ID NO: 2 - DNA, 20 bp, Photinus pyralis
ccgctggagagcaactgcat